(19) United States
(12) Patent Application Publication
(10) Pub. No.: US 2013/0247246 A1
(43) Pub. Date: Sep. 19, 2013

(54) METHOD TO REDUCE THE CONTENT OF UNSATURATED FATTY ACIDS IN LATEX RECOVERED FROM PARTHENIUM ARGENTATUM OR TARAXACUM KOK-SAGHYZ

(71) Applicant: BRIDGESTONE CORPORATION, Tokyo (JP)

(72) Inventors: Yasuyuki HAYASHI, Kodaira-shi (JP); Satoru OKUMURA, Kodaira-shi (JP)

(73) Assignee: BRIDGESTONE CORPORATION, Tokyo (JP)

(21) Appl. No.: 13/782,387

(22) Filed: Mar. 1, 2013

Related U.S. Application Data
(60) Provisional application No. 61/607,780, filed on Mar. 7, 2012.

(51) Int. Cl. C12N 15/113

(52) U.S. Cl. CPC C12N 15/1137
USPC 800/281; 800/298

(57) ABSTRACT

A method to reduce the content of unsaturated fatty acids in latex or a resin component, wherein the amount of unsaturated fatty acids in a plant body of Parthenium argentatum or Taraxacum kok-saghyz is reduced by lowering the expression level of fatty-acid desaturase in Parthenium argentatum or Taraxacum kok-saghyz by an antisense method or an RNAi method; a transgenic plant of Parthenium argentatum or Taraxacum kok-saghyz introduced with an expression vector in which a gene sequence encoding an antisense RNA or an siRNA having a nucleotide sequence complementary to an mRNA of a fatty-acid desaturase gene has been integrated into a site downstream of a promoter; and a method to reduce the content of unsaturated fatty acids in latex or a resin component, wherein the latex or the resin component is recovered from the above-mentioned transgenic plant.

### BACKGROUND OF THE INVENTION

1. Field of the Invention

The present invention relates to a method to reduce the amount of unsaturated fatty acids in latex produced from Parthenium argentatum or Taraxacum kok-saghyz.

2. Description of Related Art

Natural rubber is an elastic polymer, which is widely and abundantly used as a main ingredient of rubber products for various applications. Natural rubber is produced by collecting latex secreted from rubber trees or such latex-producing plants, and subjecting it to desired processes. For this reason, rubber trees for collecting latex, in particular, Hevea brasiliensis, are commercially planted, mainly in tropical countries such as Thailand, Malaysia, and Indonesia. Examples of such plants which contain latex (mainly, polyisoprene) in laticifers or intercellular spaces, other than Hevea brasiliensis, are: Manihot glaziovii of the family Euphorbiaceae, Ficus elastica, Castilloa elastica, and Ficus lutea Vahl of the family Moraceae; and Parthenium argentatum and Taraxacum kok-saghyz (otherwise known as Russian Dandelion) of the family Asteraceae.

In order to improve the quality of rubber products, it is effective to improve the quality of natural rubber serving as the main ingredient. For example, natural rubber containing large amounts of unsaturated fatty acids is susceptible to thermal deterioration and oxidative deterioration. Therefore, if the content of unsaturated fatty acids in latex itself collected from a latex-producing plant can be reduced, it can be expected to become possible to efficiently produce high quality rubber products.

Moreover, along with the recent development in gene engineering, character modification has become possible by introducing a preferable foreign gene into a natural plant body. Also in the field of natural rubber production, methods have been studied to make a plant having a desired character, such as a plant which can produce higher quality latex, and a plant which can produce a larger amount of latex, by genetically modifying latex-producing plants.

For example, regarding the gene transfer method in plants, mainly used methods are: the Agrobacterium method in which a gene is transferred by infecting plant cells with a bacterium belonging to the genus Agrobacterium which is a type of phytopathogenic bacterium; the particle gun method; and the electroporation method (for example, refer to Patent Document 1 and Non-patent Document 1).

Meanwhile, the amount of the production of a target substance within a plant body can be increased or decreased by enhancing or inhibiting the metabolic pathway associated with the production of the concerned substance. For example, the amount of the production of the target substance can be increased by overexpressing an enzyme associated with the metabolic pathway by transferring a gene encoding the enzyme into a plant body by means of a transformation method or the like. Conversely, the amount of the production of the target substance can be decreased by inhibiting the expression of the enzyme associated with the metabolic pathway by means of an antisense method or an RNA interference (RNAi) method. For example, it is reported that the content of unsaturated fatty acids such as linoleic acid in a plant body is increased by introducing cDNA which encodes a fatty-acid desaturase (for example, refer to Non-patent Document 1).

### Patent Documents

[Patent Document 1] Japanese Unexamined Patent Application, First Publication 2005-130815

### Non-Patent Documents

[Non-patent Document 1] Pan and four others, Plant Cell, Tissue and Organ Culture, 1996, Vol. 46, pp. 143-150

[Non-patent Document 2] Capell and one other, Current Opinion in Biotechnology, 2004, Vol. 15, pp. 148-154

[Non-patent Document 3] Ponce and one other, Rubber Chemical Technology, 1981, Vol. 54, p. 211

[Non-patent Document 4] Keller and two others, Rubber Chemical Technology, 1981, Vol. 54, pp. 115-123

### DISCLOSURE OF INVENTION

### Problems to be Solved by the Invention

Unlike Hevea brasiliensis, Parthenium argentatum does not have a so-called laticifer, but has rubber particles filled in the parenchyma of the stem, root, leaf, and the like. In general, latex is extracted by crushing the harvested stems as a whole, or such a means so as to break the parenchyma. However, in Parthenium argentatum, resin passages reside adjacent to the stem parenchyma. Therefore, the resin passages are also broken at the same time of breaking the parenchyma, which results in contamination of the extracted latex with a large amount of resin. Since the resin contains unsaturated fatty acids, the latex recovered from Parthenium argentatum has high content of unsaturated fatty acids and is thus susceptible to thermal deterioration and oxidative deterioration, which has been a problem. On the other hand, because of the recent progress in the extraction method using an organic solvent, it has become possible to separate the resin component as a by-product from the rubber component in the course of the production. As a result of investigations for the practical use of the resin component as a by-product, it has become apparent that the energy consumption for rubber kneading can be reduced by about 24% by mixing a guayule resin at about 6% during the rubber processing (for example, refer to Non-patent Document 2). However, unsaturated fatty acids contained in the resin component bring about thermal deterioration and oxidative deterioration of rubber, which has been a problem (for example, refer to Non-patent Document 3).

The present invention has been devised in view of the above-mentioned situations, with an object of providing a method to reduce the content of unsaturated fatty acids, in particular, linoleic acid, in latex recovered from Parthenium argentatum or Taraxacum kok-saghyz, or in a resin component separated as a by-product by an organic solvent extraction method.

### Means to Solve the Problems

The inventors of the present invention have conducted earnest studies to solve the above-mentioned problems. As a result, they have discovered that the content of unsaturated fatty acids in a plant body can be reduced by lowering the expression level of fatty-acid desaturase in Parthenium argentatum or Taraxacum kok-saghyz, and that latex or a resin component having low content of unsaturated fatty acids can be recovered from the Parthenium argentatum having been modified to have a low content of unsaturated fatty acids. This has led to the completion of the present invention.

That is, the object of the present invention is to provide:
(1) a method to reduce the content of unsaturated fatty acids in latex, wherein the amount of unsaturated fatty acids in a plant body of Parthenium argentatum or Taraxacum kok-saghyz is reduced by lowering the expression level of fatty-acid desaturase in Parthenium argentatum or Taraxacum kok-saghyz by an antisense method or an RNAi method.
(2) a method to reduce the content of unsaturated fatty acids in latex according to (1), wherein the expression level of fatty-acid desaturase is lowered by introducing an expression vector in which an antisense RNA or an siRNA having a nucleotide sequence complementary to an mRNA of a fatty-acid desaturase gene has been integrated into a site downstream of a promoter;
(3) a method to reduce the content of unsaturated fatty acids in latex according to (2), wherein the promoter is an expression promoter specific to rubber-synthesizing tissue and resin-synthesizing tissue;
(4) a method to reduce the content of unsaturated fatty acids in latex according to (2), wherein the promoter is an expression promoter specific to a stem;
(5) a method to reduce the content of unsaturated fatty acids in latex according to (2), wherein the promoter is an expression promoter specific to a root;
(6) a transgenic plant of Parthenium argentatum or Taraxacum kok-saghyz, introduced with an expression vector in which an antisense RNA or an siRNA having a nucleotide sequence complementary to an mRNA of a fatty-acid desaturase gene has been integrated into a site downstream of a promoter; and
(7) a method to reduce the content of unsaturated fatty acids in latex or a resin component, wherein the latex or the resin component is recovered from the transgenic plant according to (6).

### EFFECT OF THE INVENTION

In the method to reduce the content of unsaturated fatty acids in latex of the present invention, latex is recovered from a plant body whose content of unsaturated fatty acids in the plant body has been reduced by lowering the expression level of fatty-acid desaturase in Parthenium argentatum or Taraxacum kok-saghyz by an antisense method or an RNAi method. By so doing, latex having a lower content of unsaturated fatty acids can be recovered therefrom as compared to latex recovered from a plant body before lowering the expression level of fatty-acid desaturase.

### Embodiments for Carrying Out the Invention

The method to reduce the content of unsaturated fatty acids in latex of the present invention is characterized in that the amount of unsaturated fatty acids in a plant body of Parthenium argentatum or Taraxacum kok-saghyz is reduced by lowering the expression level of fatty-acid desaturase in Parthenium argentatum or Taraxacum kok-saghyz by an antisense method or an RNAi method. When the expression level of fatty-acid desaturase is lowered, it becomes difficult for the fatty acids in the plant body to be unsaturated. As a result, the content of unsaturated fatty acids is reduced.

In the present invention, the term “fatty-acid desaturase” means a polypeptide having a fatty-acid desaturase activity. In plants, the presence of three types of fatty-acid desaturases has been known as enzymes to catalyze the biosynthetic reactions from stearic acid, which is a saturated fatty acid, into oleic acid, linoleic acid, or lionlenic acid, which are unsaturated fatty acids, namely: Δ9 fatty-acid desaturase (for example, NCBI (National Center for Biotechnology Information) Accession Number: D88536 in the case of Arabidopsis thaliana); Δ12 fatty-acid desaturase (for example, NCBI Accession Number: AF251844, in the case of Helianthus annuus); and ω3 fatty-acid desaturase (for example, NCBI Accession Number: AY254858, in the case of Helianthus annuus).

The expression level of fatty-acid desaturase in a plant body can be lowered by an antisense method or an RNAi method. The antisense method is a method to inhibit the mRNA translation of a target gene whose expression level is desired to be lowered, by introducing a single stranded antisense RNA having a nucleotide sequence that is complementary to the mRNA of the target gene in vivo. On the other hand, the RNAi method is a method to inhibit the mRNA translation of the target gene by introducing a double stranded RNA having a nucleotide sequence that is homologous with the whole or a part of the mRNA of the target gene in vivo.

Specifically speaking, a single stranded antisense RNA having a nucleotide sequence that is complementary to the mRNA of the fatty-acid desaturase gene (hereunder, referred to as the “antisense RNA of the fatty-acid desaturase gene”), or a double stranded siRNA having a nucleotide sequence that is homologous with the whole or a part of the mRNA of the fatty-acid desaturase gene (hereunder, referred to as the “siRNA of the fatty-acid desaturase gene”) is introduced into the plant body of Parthenium argentottim. Here, the term “fatty-acid desaturase gene” means a gene which encodes a polypeptide having a fatty-acid desaturase activity.

The antisense RNA of the fatty-acid desaturase gene may be either complementary to the whole of the mRNA of the fatty-acid desaturase gene, or complementary to a part of the mRNA of the concerned gene, as long as the antisense RNA has an antisense effect. Moreover, the antisense RNA of the fatty-acid desaturase gene suffices if it is a single stranded RNA that is hybridizable with the mRNA of the fatty-acid desaturase gene, and it may either be completely complementary to the mRNA of the fatty-acid desaturase gene or contain partial mismatch(es).

The siRNA of the fatty-acid desaturase gene is a double stranded RNA of about 21 base pairs consisting of: an antisense strand having a nucleotide sequence that is complementary to the mRNA of the fatty-acid desaturase gene; and a sense strand composed of a nucleotide sequence that is complementary to the antisense strand; with a structure in which the 3′ end of each RNA strand has two overhanging nucleotides. The siRNA suffices if it is complementary to a part of the mRNA of the fatty-acid desaturase gene, as long as the siRNA has an RNA interference effect. Moreover, the siRNA of the fatty-acid desaturase gene may either be completely homologous with the mRNA of the fatty-acid desaturase gene or contain partial mismatch(es), as long as the siRNA has an RNA interference effect.

In the present invention, the phrase “the antisense RNA has an antisense effect” means that the expression level of fatty-acid desaturase is lowered as a result of the introduction of the antisense RNA into the plant body. Similarly, the phrase “the siRNA has an RNA interference effect” means that the expression level of fatty-acid desaturase is lowered as a result of the introduction of the siRNA into the plant body.

The design of the nucleotide sequence of the antisense RNA or the siRNA of the fatty-acid desaturase gene (in other words, the design regarding which region of the mRNA of the fatty-acid desaturase gene the nucleotide sequence should be homologous with) and the synthesis thereof can be done by any known method in this technical field. For example, the antisense RNA or the siRNA is preferably designed so as to have a nucleotide sequence complementary to a region in the vicinity of the start codon from the ribosome binding site of the mRNA of the fatty-acid desaturase gene, because the expression level of fatty-acid desaturase can be more effectively lowered. In addition, the antisense RNA or the siRNA of the fatty-acid desaturase gene to be introduced into the plant body preferably takes a form of a united nucleic acid incorporated with another nucleotide sequence that is useful for exerting the function to inhibit the translation of the mRNA of the fatty-acid desaturase gene within the plant body. In particular, as a standard of the design of the siRNA of the fatty-acid desaturase gene, it is desirable to fulfill the conditions in which the 5′ end of the guide strand is either A or U, the 5′ end of the passenger strand is either G or C, the 5′ region of the antisense strand is rich in A or U, and a long and contiguous sequence of GC is not present.

The introduction of the antisense RNA or the siRNA of the fatty-acid desaturase gene into the plant body may be either transient or constitutive. For example, even in a case where the antisense RNA or the siRNA has been directly introduced into the plant body by means of an electroporation method, a particle gun method, or the like, the expression level of fatty-acid desaturase is able to be lowered in a site where the antisense RNA or the siRNA has been introduced.

In the present invention, it is preferable to use an expression vector because it is relatively easy to handle and it can be stably introduced into the plant body. Specifically speaking, the expression level of fatty-acid desaturase can be lowered by introducing an expression vector in which the antisense RNA or the siRNA of the fatty-acid desaturase gene has been integrated into a site downstream of a promoter.

The expression vector for use in the present invention may be any known expression vector for use in the production of transgenic plants, and can be appropriately selected according to the method to introduce it into Parthenium argentatum or Taraxacum kok-saghyz. Moreover, it is also possible to use a vector produced by modifying a known vector. In addition, it is also preferable to use a commercially available siRNA vector.

The expression vector in which the antisense RNA or the siRNA of the fatty-acid desaturase gene has been integrated may be produced by any known method in this field. In general, the antisense RNA or the siRNA is integrated into a vector in a state where transcriptional and translational regulatory regions such as a promoter, a terminator, and the like, are held at the beginning and the end of the DNA which constitutes the gene as a target to be introduced into the plant body. The genes of these regulatory regions suffice as long as they can exert their functions in the plant where the genes are introduced, and may be either genes derived from Parthenium argentatum or from another creature belonging to a different species. Regarding such a promoter of a different species, it is possible to use, for example, a general purpose promoter in the field associated with gene recombination such as the CaMV35S promoter and the NOS promoter.

In Parthenium argentatum, the latex, in particular, the resin component, is mainly present in the parenchyma of the stem and root. For this reason, in the present invention, regarding the promoter to be arranged on the upstream of the antisense RNA or the siRNA of the fatty-acid desaturase gene, it is preferable to use a promoter specific to rubber-synthesizing tissue and resin-synthesizing tissue, an expression promoter specific to the stem, or an expression promoter specific to the root. The influence on the metabolism of the whole plant body due to the suppression of the expression of the fatty-acid desaturase can be reduced by exclusively lowering the expression level of fatty-acid desaturase only in the tissues of the plant body from which the latex can be recovered. The term “expression promoter specific to rubber-synthesizing tissue, stem, or root” means a promoter to express the gene on the downstream of the promoter specifically in the rubber-synthesizing tissues, stems, or roots, within the plant body. Moreover, in the present invention, the term “stem” suffices if it is a site where the cambium is formed, and it may be the trunk of a mature tree.

In addition, the expression vector for use in the present invention may be a vector in which only the antisense RNA or the siRNA of the fatty-acid desaturase gene is integrated, or a vector in which other gene(s) is(are) also integrated. For example, the antisense RNA or the siRNA of the fatty-acid desaturase gene may be integrated into the expression vector together with a selection marker gene serving as an index to indicate the success or failure of the introduction of the expression vector. The selection marker can be exemplified by a drug resistance gene such as the kanamycin-resistance gene (nptII), the hygromycin resistance gene (hptI), and the bialaphos resistance gene (bar). In addition, it is also preferable to use a gene which encodes a fluorescent protein as a selection marker. The fluorescent protein can be appropriately selected from among usual fluorescent proteins for use in the integration into a protein expression vector or the like, such as a green fluorescent protein (GFP), a yellow fluorescent protein (YFP), a red fluorescent protein (RFP), ZsGreen1, and DsRed.

The method to produce a transgenic plant by introducing the expression vector having the thus integrated antisense RNA or the siRNA of the fatty-acid desaturase gene into Parthenium argentatum, is not specifically limited, and any known technique to be used for producing a plant transformant may be adopted. For example, a transgenic plant in which the antisense RNA or the siRNA of the fatty-acid desaturase gene has been introduced can be produced by introducing the expression vector into Parthenium argentatum by using various types of known methods in this technical field, such as the Agrobacterium method, the particle gun method, the electroporation method, and the like.

In the present invention, it is preferable to produce a transgenic plant by using the Agrobacterium method. This is because Parthenium argentatum shows high infection efficiency with a bacterium of Agrobacterium and also has excellent redifferentiation efficiency. Thus, Parthenium argentatum is suitable for the production of transgenic plants by means of the Agrobacterium method.

The Agrobacterium method is a method to introduce a gene in a plant body such that: an expression vector integrated with a gene to be introduced into the plant body is transferred in a bacterium belonging to the genus Agrobacterium; this bacterium belonging to the genus Agrobacterium having the thus transferred gene is cultured to effect proliferation by a conventional method; and thereafter a callus or a young plant body is infected with the bacterium. The bacterium belonging to the genus Agrobacterium including the expression vector may be produced by using any conventionally known technique. For example, the technique may be such that: a gene recombinant intermediate vector in which the antisense RNA or the siRNA of the fatty-acid desaturase gene has been integrated into a plasmid capable of homologous recombination with the T-DNA region of the Ti plasmid held by a bacterium belonging to the genus Agrobacterium is produced; and this gene recombinant intermediate vector is introduced into the bacterium belonging to the genus Agrobacterium. In this case, this gene recombinant intermediate vector corresponds to the expression vector of the present invention. In addition, the procedure may also be such that: a recombinant binary vector in which the antisense RNA or the siRNA of the fatty-acid desaturase gene has been integrated into a general purpose binary vector for the Agrobacterium method, is introduced into a bacterium belonging to the genus Agrobacterium. In this case, this recombinant binary vector corresponds to the expression vector of the present invention. The bacterium belonging to the genus Agrobacterium for use in the infection is not specifically limited, as long as it is a bacterium belonging to the genus Agrobacterium which is capable of introducing a vector such as a plasmid held therein into plant cells. Preferred is Agrobacterium tumefaciens. This is because it offers excellent infection efficiency and has been used for general purposes in the Agrobacterium method.

The transgenic plant of Parthenium argentatum or Taraxacum kok-saghyz can be obtained by, for example, bringing a tissue such as a root, a stem, a leaf, or a flower, or cells such as a callus, of Parthenium argentatum or Taraxacum kok-saghyz into contact with an infection solution containing the above-mentioned expression vector, followed by tissue culture. If a drug resistance gene is used as a selection marker, it is possible to efficiently select the transgenic plant by additional culture in a selection medium containing an antibiotic.

The tissue or cells are supplied to the tissue culture after disinfection or sterilization. The disinfection or sterilization may be performed by using a known disinfectant or sterilization agent. It is preferable to use, for example, ethanol, benzalkonium chloride, sodium hypochlorite solution, or the like.

The tissue culture may be performed under a light shielding condition, preferably at 18 to 30° C., and more preferably at 25 to 28° C., preferably for about 2 to 4 days, on a medium of the same composition.

The culture on the selection medium is performed by transferring pieces of the transgenic tissue to a fresh medium, preferably every 20 to 30 days. The culture temperature may be the same as the tissue culture mentioned above.

The transgenic plant selected by the selection medium can be kept cultured on a redifferentiation medium so as to elongate shoots. Roots can be developed from the elongated shoots. By so doing, a young plant body can be obtained. Thereafter, this young plant is transplanted into soil for acclimation, and grown preferably at 18 to 30° C., and more preferably at 28 to 30° C. By so doing, a mature transgenic plant can be obtained.

The thus obtained transgenic plant can be cultivated and the latex can be recovered therefrom, in the same manner as that for Parthenium argentatum or Taraxacum kok-saghyz before the introduction of the expression vector. Moreover, the produced transgenic plant has a lower expression level of fatty-acid desaturase and a reduced content of unsaturated fatty acids, as compared to Parthenium argentatum or Taraxacum kok-saghyz before the introduction of the expression vector. For this reason, latex or a resin component having a low content of unsaturated fatty acids with improved thermal deterioration resistance and oxidative deterioration resistance can be recovered from this transgenic plant.

### EXAMPLES

Next is a more detailed description of the present invention with reference to Examples. However, the present invention is not to be limited to the following Examples.

### Example 1

An siRNA of the fatty-acid desaturase gene was introduced into Parthenium argentatum, and the amount of unsaturated fatty acids in the plant body was investigated.

<Construction of siRNA Vector>

A product made by ligating a gene sequence encoding an siRNA which included an antisense strand having a nucleotide sequence homologous with the sequence from the 19th base to the 39th base of the mRNA of stearoyl-ACP desaturase gene of Helianthus annuus (NCBI Accession Number: U91340) to the 3′ side of the 2 kbp sequence on the 5′ upstream side of the genome of a gene related to FPP synthase of Parthenium argentatum (NCBI Accession Number: 35935), was integrated into a binary vector pB1121 for both Escherichia coli and Agrobacterium by a conventional method. This pBI121 vector integrated with the siRNA was used as an siRNA-containing expression vector. This siRNA-containing expression vector was able to express the siRNA in tissues where the biosynthesis of rubber was performed, by a promoter for the gene related to rubber synthesis of Parthenium argentatum in the plant body as a result of the introduction. Escherichia coli introduced with the siRNA-containing expression vector was cultured to prepare the siRNA-containing expression vector, and this vector was introduced into Agrobacterium LBA4404.

<Production of Transgenic Parthenium argentatum>

Using the Agrobacterium having been introduced with the siRNA-containing expression vector, a leaf of Parthenium argentatum as a material was subjected to transfection. By so doing, a transgenic plant of Parthenium argentatum (transgenic Parthenium argentatum) in which the siRNA of the fatty-acid desaturase gene was expressed in rubber-synthesizing tissues, was produced.

<Analysis of Fatty Acids in Transgenic Parthenium argentatum>

Latex is extracted from the obtained transgenic Parthenium argentatum, and coagulated to obtain rubber. The obtained rubber was subjected to acetone extraction, and the extracted matter was analyzed for fatty acids according to an ordinary method (refer to Non-patent Document 3). As a control, Parthenium argentatum without the introduction of the siRNA-containing expression vector (non transgenic plant) was also analyzed for fatty acids in the same manner.

It was found that the proportion of unsaturated fatty acids in the total fatty acids contained in the latex was 80% or higher in the case of the non transgenic plant, while this proportion could be reduced to 10 to 40% in the case of the transgenic Parthenium argentatum.

### Example 2

<Construction of siRNA Vector>

A product made by ligating a gene sequence encoding an siRNA which included an antisense strand having a nucleotide sequence homologous with the sequence from the 550th base to the 570th base of the mRNA of the fatty-acid desaturase gene (NCBI Accession Number: AF254858) to the same as that of Example 1, was integrated into a binary vector pBI121 for both Escherichia coli and Agrobacterium by a conventional method. Using the thus produced Agrobacterium having been introduced with the siRNA-containing expression vector, a leaf of Parthenium argentatum as a material was subjected to transfection, and the thus obtained transgenic Parthenium argentatum was analyzed for fatty acids, by the same procedures as those of Example 1.

### Example 3

<Construction of siRNA Vector>

A product made by ligating a gene sequence encoding an siRNA which included an antisense strand having a nucleotide sequence homologous with the sequence from the 343th base to the 363th base of the mRNA of the fatty-acid desaturase gene (NCBI Accession Number: D88536) to the same as that of Example 1, was integrated into a binary vector pBI121 for both Escherichia coli and Agrobacterium by a conventional method. Using the thus produced Agrobacterium having been introduced with the siRNA-containing expression vector, a leaf of Parthenium argentatum as a material was subjected to transfection, and the thus obtained transgenic Parthenium argentatum was analyzed for fatty acids, by the same procedures as those of Example 1.

### Example 4

An antisense RNA of the fatty-acid desaturase gene was introduced into Parthenium argentatum, and the amount of unsaturated fatty acids in the plant body was investigated.

<Construction of siRNA Vector>

A product made by ligating a nucleotide sequence from the 19th base to the 464th base of the mRNA of the fatty-acid desaturase gene (NCBI Accession Number: AF251844) in the antisense direction to the 3′ side of the 2 kbp sequence on the 5′ upstream side of the genome of a gene related to rubber synthesis of Parthenium argentatum (NCBI Accession Number: AF541942), was integrated into a binary vector pBI121 for both Escherichia coli and Agrobacterium by a conventional method. This pBI121 vector integrated with the antisense RNA was used as an antisense RNA-containing expression vector. This antisense RNA-containing expression vector was able to express the antisense RNA in tissues where the biosynthesis of rubber was performed, by a promoter for a rubber synthesis-related gene of Parthenium argentatum in the plant body as a result of the introduction. Escherichia coli introduced with the antisense RNA-containing expression vector was cultured to prepare the antisense RNA-containing expression vector, and this vector was introduced into Agrobacterium LBA4404.

<Production of Transgenic Parthenium argentatum>

Using the Agrobacterium having been introduced with the antisense RNA-containing expression vector, a leaf of Parthenium argentatum as a material was subjected to transfection. By so doing, a transgenic plant of Parthenium argentatum (transgenic Parthenium argentatum), in which the antisense RNA of the fatty-acid desaturase gene was expressed in rubber-synthesizing tissues, was produced.

<Analysis of Fatty Acids in Transgenic Parthenium argentatum>

Latex is extracted from the obtained transgenic Parthenium argentatum, and coagulated to obtain rubber. The obtained rubber was subjected to acetone extraction, and the extracted matter was analyzed for fatty acids according to an ordinary method. As a control, Parthenium argentatum without the introduction of the antisense RNA-containing expression vector (non transgenic plant) was also analyzed for fatty acids in the same manner.

It was found that the proportion of unsaturated fatty acids in the total fatty acids contained in the latex was 80% or higher in the case of the non transgenic plant, while this proportion could be reduced to about 40% or lower in the case of the transgenic Parthenium argentatum.

### Example 5

### <Construction of Antisense RNA Vector>

A product made by ligating a nucleotide sequence from the 550th base to the 780th base of the mRNA of the fatty-acid desaturase gene (NCBI Accession Number: AF254858) in the antisense direction to the same as that of Example 4, was integrated into a binary vector pBI121 for both Escherichia coli and Agrobacterium by a conventional method. Using the thus produced Agrobacterium having been introduced with the antisense RNA-containing expression vector, a leaf of Parthenium argentatum as a material was subjected to transfection, and the thus obtained transgenic Parthenium argentatum was analyzed for fatty acids, by the same procedures as those of Example 4.

### Example 6

### <Construction of Antisense RNA Vector>

A product made by ligating a nucleotide sequence from the 233th base to the 536th base of the mRNA of the fatty-acid desaturase gene (NCBI Accession Number: D88536) in the antisense direction to the same as that of Example 4, was integrated into a binary vector pBI121 for both Escherichia coli and Agrobacterium by a conventional method. Using the thus produced Agrobacterium having been introduced with the antisense RNA-containing expression vector, a leaf of Parthenium argentatum as a material was subjected to transfection, and the thus obtained transgenic Parthenium argentatum was analyzed for fatty acids, by the same procedures as those of Example 4.

### INDUSTRIAL APPLICABILITY

According to the method to reduce the content of unsaturated fatty acids in latex of the present invention, a higher quality of latex or resin components can be obtained by reducing the content of unsaturated fatty acids in latex or a resin component, recovered from Parthenium argentatum or Taraxacum kok-saghyz. Therefore, the present invention is particularly useful in the field of natural rubber production.

1. A method to reduce the content of unsaturated fatty acids in latex, wherein the amount of unsaturated fatty acids in a plant body of Parthenium argentatum or Taraxacum kok-saghyz is reduced by lowering the expression level of fatty-acid desaturase in Parthenium argentatum or Taraxacum kok-saghyz by an antisense method or an RNAi method.

2. A method to reduce the content of unsaturated fatty acids in latex according to claim 1, wherein the expression level of fatty-acid desaturase is lowered by introducing an expression vector in which a gene sequence encoding an antisense RNA or an siRNA having a nucleotide sequence complementary to an mRNA of a fatty-acid desaturase gene has been integrated into a site downstream of a promoter.

3. A method to reduce the content of unsaturated fatty acids in latex according to claim 2, wherein said promoter is an expression promoter specific to rubber-synthesizing tissue and resin-synthesizing tissue.

4. A method to reduce the content of unsaturated fatty acids in latex according to claim 2, wherein said promoter is an expression promoter specific to a stem.

5. A method to reduce the content of unsaturated fatty acids in latex according to claim 2, wherein said promoter is an expression promoter specific to a root.

6. A transgenic plant of Parthenium argentatum or Taraxacum kok-saghyz, introduced with an expression vector in which a gene sequence encoding an antisense RNA or an siRNA having a nucleotide sequence complementary to an mRNA of a fatty-acid desaturase gene has been integrated into a site downstream of a promoter.

7. A method to reduce the content of unsaturated fatty acids in latex or a resin component, wherein the latex or the resin component is recovered from the transgenic plant according to claim 6.
